(19) United States
(12) Patent Application Publication
(10) Pub. No.: US 2012/0294857 A1
(43) Pub. Date: Nov. 22, 2012

(54) Monomeric Bi-Specific Fusion Protein

(75) Inventors: Charles L. Sentman, West Lebanon, NH (US); Tong Zhang, Beijing (CN)

(73) Assignee: Trustees of Dartmouth College, Hanover, NH (US)

(21) Appl. No.: 13/521,532

(22) PCT Filed: Jan. 7, 2011
(86) PCT No.: PCT/US11/20490

§ 371 Date: Jul. 11, 2012

Related U.S. Application Data
(60) Provisional application No. 61/293,904, filed on Jan. 11, 2010.

(51) Int. Cl. C07K 19/00; C12N 15/62; A61P 31/00; C12N 1/21; C12N 5/10; A61P 35/00; A61K 39/395; C12N 15/63

(52) U.S. Cl. 424/134.1; 530/387.3; 536/23.4; 435/320.1; 435/252.3; 435/328

(57) ABSTRACT

The present invention embraces a bi-specific fusion protein composed of an effector cell-specific antibody-variable region fragment operably linked to at least a portion of a natural killer cell receptor. Methods for using the fusion protein in the treatment of cancer and pathogenic infections are also provided.

### INTRODUCTION

This application claims benefit of priority to U.S. Provisional Application Ser. No. 61/293,904, filed Jan. 11, 2010, the content of which is incorporated herein by reference in its entirety.

The research underlying this invention was supported in part with funds from National Institutes of Health Grant Nos. R0l CA130911 and T32 AR007576. The United States Government has certain rights in this invention.

### BACKGROUND OF THE INVENTION

T cells, especially cytotoxic T cells, play important roles in anti-tumor immunity (Rossing and Brenner (2004) Mol. Ther. 10:5-18). Adoptive transfer of tumor-specific T cells into patients provides a means to treat cancer (Sadelain, et al. (2003) Nat. Rev. Cancer 3:35-45). However, the traditional approaches for obtaining large numbers of tumor-specific T cells are time-consuming, laborious and sometimes difficult because the average frequency of antigen-specific T cells in periphery is extremely low (Rosenberg (2001) Nature 411:380-384; Ho, et al. (2003) Cancer Cell 3:431-437; Crowley, et al. (1990) Cancer Res. 50:492-498). In addition, isolation and expansion of T cells that retain their antigen specificity and function can also be a challenging task (Sadelain, et al. (2003) supra). Genetic modification of primary T cells with tumor-specific immunoreceptors, such as full-length T cell receptors or chimeric T cell receptor molecules can be used for redirecting T cells against tumor cells (Stevens, et al. (1995) J. Immunol. 154:762-771; Oelke, et al. (2003) Nat. Med. 9:619-624; Stancovski, et al. (1993) J. Immunol. 151:6577-6582; Clay, et al. (1999) J. Immunol. 163:507-153). This strategy avoids the limitation of low frequency of antigen-specific T cells, allowing for facilitated expansion of tumor-specific T cells to therapeutic doses.

Natural killer (NK) cells are innate effector cells serving as a first line of defense against certain viral infections and tumors (Biron, et al. (1999) Annu. Rev. Immunol. 17:189-220; Trinchieri (1989) Adv. Immunol. 47:187-376). They have also been implicated in the rejection of allogeneic bone marrow transplants (Lanier (1995) Curr. Opin. Immunol. 7:626-631; Yu, et al. (1992) Annu. Rev. Immunol. 10:189-214). Innate effector cells recognize and eliminate their targets with fast kinetics, without prior sensitization. Therefore, NK cells need to sense if cells are transformed, infected, or stressed to discriminate between abnormal and healthy tissues. According to the missing self phenomenon (Kärre, et al. (1986) Nature (London) 319:675-678), NK cells accomplish this by looking for and eliminating cells with aberrant major histocompatibility complex (MHC) class I expression; a concept validated by showing that NK cells are responsible for the rejection of the MHC class I-deficient lymphoma cell line RMA-S, but not its parental MHC class I-positive line RMA.

Inhibitory receptors specific for MHC class I molecules have been identified in mice and humans. The human killer cell Ig-like receptors (KIR) recognize HLA-A, -B, or -C; the murine Ly49 receptors recognize H-2K or H-2D; and the mouse and human CD94/NKG2 receptors are specific for Qa1^{b} or HLA-E, respectively (Long (1999) Annu. Rev. Immunol. 17:875-904; Lanier (1998) Annu. Rev. Immunol. 16:359-393; Vance, et al. (1998) J. Exp. Med. 188:1841-1848).

Activating NK cell receptors specific for classic MHC class I molecules, nonclassic MHC class I molecules or MHC class I-related molecules have been described (Bakker, et al. (2000) Hum. Immunol. 61:18-27). One such receptor is NKG2D (natural killer cell group 2D) which is a C-type lectin-like receptor expressed on NK cells, yδ-TcR⁺ T cells, and CD8⁺ αβ-TcR⁺ T cells (Bauer, et al. (1999) Science 285:727-730). NKG2D is associated with the transmembrane adapter protein DAP10 (Wu, et al. (1999) Science 285:730-732), whose cytoplasmic domain binds to the p85 subunit of the PI-3 kinase.

In humans, two families of ligands for NKG2D have been described (Bahram (2000) Adv. Immunol. 76:1-60; Cerwenka and Lanier (2001) Immunol. Rev. 181:158-169). NKG2D binds to the polymorphic MHC class I chain-related molecules (MIC)-A and MIC-B (Bauer, et al. (1999) supra). These are expressed on many human tumor cell lines, on several freshly isolated tumor specimens, and at low levels on gut epithelium (Groh, et al. (1999) Proc. Natl. Acad. Sci. USA 96:6879-6884). NKG2D also binds to another family of ligands designated the RAET-1 family or UL binding proteins (ULBP)-1, -2, -3, and -4 molecules (Cosman, et al. (2001) Immunity 14:123-133; Kubin, et al. (2001) Eur. J. Immunol. 31:1428-1437). Although similar to class I MHC molecules in their α1 and α2 domains, the genes encoding these proteins are not localized within the MHC. Like MIC (Groh, et al. (1996) supra), the ULBP molecules do not associate with β₂-microglobulin or bind peptides. The known murine NKG2D-binding repertoire encompasses the retinoic acid early inducible-1 gene products (RAE-1) and the related H60 minor histocompatibility antigen (Cerwenka, et al. (2000) Immunity 12:721-727; Diefenbach, et al. (2000) Nat. Immunol. 1:119-126). RAE-1, Mult-1, and H60 were identified as ligands for mouse NKG2D by expression cloning these cDNA from a mouse transformed lung cell line (Cerwenka, et al. (2000) supra). Transcripts of RAE-1 are rare in adult tissues but abundant in the embryo and on many mouse tumor cell lines, indicating that these are oncofetal antigens.

Molecules which target both effector lymphocytes and tumor cells have been suggested. For example, U.S. Patent Application No. 2008299137 suggests a dimeric fusion protein composed of an antibody-like protein that is specific for an activating receptor on an effector lymphocyte linked to a portion of a cell membrane protein that binds to a cell-associated target.

### SUMMARY OF THE INVENTION

The present invention features a monomeric bi-specific fusion protein composed of an effector cell-specific antibody fragment consisting of the variable region (Fv), which is operably linked, e.g., via a linker, to at least a portion of a natural killer cell receptor, wherein in one embodiment the portion of the natural killer cell receptor is the extracellular domain. In some embodiments the NK cell receptor is selected from NKG2D, NKG2A/CD94, NKRPl, NKG2C/CD94, NKG2E/CD94, NKG2F/CD94, NKp30, NKp44, NKp46, DNAM-1, CD69, LLT1, AICL, and CD26. In other embodiments the Fv region binds an activating receptor expressed on a T cell, NK cell, macrophage, dendritic cell, or neutrophil. In particular embodiments, the activating receptor is selected from CD3, CD4, CD8, CD16, CD28, CD16, NKp30, NKp44, NKp46, mannose receptor, CD64, scavenger receptor A, and DEC205. Pharmaceutical compositions containing the fusion protein and optionally at least one second therapeutic agent are also provided as are nucleic acid molecules encoding the fusion protein, and a vector and host cell containing the same.

The present invention also features methods for preventing or treating cancer; enhancing immunity against a tumor; and treating a pathogen infection by administering to a subject in need of treatment an effective amount of a fusion protein of the invention.

### BRIEF DESCRIPTION OF THE DRAWINGS

FIG. 1 depicts the structure of scFvscFv-NKG2D. Anti-CD3ε V_{H} and V_{L} are linked with G4S linker (L₁) and fused to the extracellular domain (Ex) of NKG2D receptor with the second G4S linker (L₂) in between. For the convenience of protein purification, a histidine tag (6 repeats of histidine) was added at the C-terminus.

FIG. 2 shows that T cells respond to NKG2D ligand-positive cells by producing IFN-γ in the presence of scFv-NKG2D. Bulk spleen cells were stimulated with ConA (1 μg/ml) and IL-2 (25 U/ml) before co-culture with irradiated tumor cells for 24 hours. Both suspension (FIG. 2A) (10⁵) and adherent tumor cells (FIG. 2B) (2.5×10⁴) were co-cultured with ConA-stimulated spleen cells (10⁵) in 96-well plates. The scFv-NKG2D was added at 50 ng/ml. Fusion protein scFv-huNKG2D (in which mouse NKG2D was replaced with the extracellular domain of human NKG2D) was used as a negative control. IFN-γ amounts in the supernatants were analyzed with ELISA. Results are shown in mean ±SD.

FIG. 3 shows that T cells can kill NKG2D ligand-positive tumor cells in the presence of scFv-NKG2D. ConA-stimulated T cells were co-cultured with NKG2D ligand-positive P815/Rae1 cells at E:T ratios of 1:1 to 25:1 in the presence (closed symbols) or absence (open symbols) of scFv-NKG2D, for 5 hours. The specific lysis was determined by Cr⁵¹-assay. The scFv-NKG2D was added as conditioned media (CM), which was produced by cells stably transfected with scFv-NKG2D. Results are shown in mean ±SD.

FIG. 4 shows that scFv-NKG2D expression in MC-38 cells reduces or abrogate tumor growth. FIG. 4A, Mouse colon cancer MC-38 cells were genetically modified with a retroviral vector containing either scFv-mNKG2D (open square, n=22) or control molecule scFv-HuNKG2D (filled triangle, n=12) and then injected s.c. (5×10⁵) into right flanks of B6 mice on day 0. Only 9 of 22 mice developed tumors, whereas in HBSS-treated groups (filled square, n=19) in which wild-type MC-38 cells were given, all 19 mice developed tumors. The tumor areas are pooled data from four independent experiments. FIG. 4B, Intravenous administration of purified scFv-NKG2D promotes survival in a systemic lymphoma model. Treatment of RMA/RG (10⁵, i.v., day 0) tumor-bearing mice with 3 doses of scFv-mNKG2D (open diamond, 5 μg, i.v. n=19) on days 5, 7 and 9 significantly enhanced survival compared to HBSS (filled square, n=19) or control molecule scFv-HuNKG2D (n=8). Data are presented in Kaplan-Meier survival curves. *: p<0.002. FIG. 4C, Tumor free mice (open circle) in the MC-38/scFv-NKG2D group (shown in FIG. 4A) and age-matched naïve mice (filled square) were re-challenged with wild-type MC-38 cells (10⁵) s.c. into the left flanks. FIG. 4D, Tumor free mice (open circle) in the scFv-NKG2D-treated RMA lymphoma model (shown in FIG. 4B) and age-matched naïve mice (filled square) were re-challenged with wild type RMA cells (10⁴) s.c. into the left flanks. The tumor areas are represented as Mean+SEM. The error bars represent SEM.

FIG. 5 shows that T cells can kill NKp30 ligand-positive cells in the presence of NKp30-scFv. FIG. 5A, Anti-CD3-stimulated T cells were co-cultured with NKp30 ligand-negative mouse cell RMA and an NKp30 ligand B7-H6-transduced RMA (RMA/B7-H6) at an E:T ratio of 10:1 in the presence (filled bars) or absence (open bars) of NKp30-scFv (50 ng/ml) for 5 hours. The specific lysis was determined by a LDH-release assay. Results are shown in mean+SD of triplicates. FIG. 5B, A dose-response was determined. The specific lysis was determined after adding varying concentrations of NKp30-scFv (0-160 ng/ml) to the co-culture of T cells and tumor cells.

FIG. 6 shows that T cells respond to NKp30 ligand-positive cells by producing IFN-γ in the presence of NKp30-scFv. Human PBMCs were stimulated with anti-CD3 (140 ng/ml) and IL-2 (50 U/ml) before co-culture with mitomycin C-treated tumor cells for 24 hours. T cells (10⁵) were incubated with 10⁵ RMA (ligand-negative), RMA/B7-H6 (ligand-positive) or K562(ligand-positive) in 96-well plates in the presence (filled) or absence (open) of NKp30-scFv (50 ng/ml). IFN-γ amounts in the supernatants were analyzed with ELISA. Results are shown in mean +SD. These data show that the expression of NKp30 ligands on tumor cells is required for induction of IFN-γ production. *: P<0.01 (NKp30-scFv vs media).

### DETAILED DESCRIPTION OF THE INVENTION

A monomeric bi-specific fusion protein has now been developed that is composed of two different binding sites. One binding site is an antibody variable fragment region (Fv) specific for an effector cell, and the other site is at least a portion of a NK receptor molecule. This fusion protein can indirectly decrease tumor growth or exert anti-pathogen effects by, e.g., inducing the expression or activity of cytokines, or other soluble factors, which results in the activation of immune cells or inhibition of local immunosuppressive cells such as Tregs or MDSCs. Alternatively, this bi-specific molecule (containing two active binding sites) can directly engage effector cells and a target cell, such as a cancer cell. In turn, the activated T cell releases effector functions against the bound tumor cell thereby resulting in death of the target cell. Thus, this invention is a novel means to target tumor cells using NK cell receptors to guide effector cells to tumor cells. Due to the nature of ligand expression on many different types of tumor cells, the instant bi-specific fusion proteins are useful against many types of tumor cells. Because certain ligands can be expressed by virus- or bacterial-infected cells, the instant fusion proteins can also be used in targeting cells infected by pathogens.

A bi-specific fusion protein of the invention is monomeric in the sense that it is produced with components that do not have a tendency to dimerize with another fusion protein of the invention. In this respect, the instant fusion protein does not self-associate into a polypeptide possessing two associated components which form a dimer.

According to the present invention, an effector cell-specific antibody variable region fragment is intended to mean a fragment of an antibody consisting of the variable domain (i.e., Fv region), which specifically binds to an effector cell activating receptor. In this respect, the antibody Fv region of the present bispecific molecule does not include the Fc (Fragment, crystallizable) region of the antibody. While inclusion of the Fc region of an antibody may facilitate retention in the body, not wishing to be bound by theory, it is believed that were the Fc region included in the instant bispecific molecule, the Fc region would interfere with the mode of action.

The structure of an antibody is well-known in the art. See, e.g., Fundamental Immunology (Paul, W., ed., 2nd ed. Raven Press, N.Y. (1989)). The numbering of amino acid residues in the variable region of a naturally occurring antibody (which includes the complementarity determining regions (CDRs) interspersed with the conserved framework regions (FR)) can be conveniently performed using the method described in Kabat et al., Sequences of Proteins of Immunological Interest, 5th Ed. Public Health Service, National Institutes of Health, Bethesda, Md. (1991). Using this numbering system, the actual linear amino acid sequence of a peptide may contain fewer or additional amino acids corresponding to a shortening of, or insertion into, a CDR of the variable domain. For example, a heavy chain variable domain may include a single amino acid insert (residue 52a according to Kabat) after residue 52 of CDR H2. The Kabat numbering of residues may be determined for a given antibody by alignment at regions of identity of the sequence of the antibody with a “standard” Kabat numbered sequence.

Unless otherwise stated or indicated, the term “antibody” herein includes polyclonal antibodies and monoclonal antibodies (mAbs). The term “monoclonal antibody” refers to a homogeneous antibody population having a uniform structure and specificity. Polyclonal antibodies have mixed specificity. Polyclonal antibodies typically are derived from the serum of an animal that has been immunogenically challenged. Monoclonal antibodies can be produced by various known means, such as through hybridoma technology, phage display technology, or synthesis methods, examples of which are known in the art.

An antibody in the context of this invention can possess any isotype and an antibody of interest of a particular isotype can be “isotype switched” with respect to an original antibody from which it is derived using conventional techniques. Such techniques include the use of direct recombinant techniques (see e.g., U.S. Pat. No. 4,816,397), cell-cell fusion techniques (see e.g., U.S. Pat. No. 5,916,771), and other suitable techniques known in the art. Typically, an Fv region of the invention is derived from an IgG isotype antibody.

The Fv region of an antibody can be obtained by actual fragmentation of an antibody molecule, by recombinant production, or by another suitable technique. For example, the Fv region, consisting essentially of the VL and VH domains of a single arm of an antibody, can be generated by expression of nucleic acids encoding said region in recombinant cells (see, e.g., Evans, et al. (1995) J. Immunol. Meth. 184:123-38). Moreover, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain antibodies or single chain Fv (scFv); see e.g., Bird, et al. (1988) Science 242:423-426, and Huston, et al. (1988) Proc. Natl. Acad. Sci. USA 85:5879-5883).

The Fv region of the instant bispecific molecule can be of any suitable length and composition, as long as it is capable of specifically binding to a receptor of an effector cell. Typically, an Fv region is about 50-350 amino acids in length, or more desirably 100-300 amino acids, in length.

In one embodiment, the Fv region does not itself activate the effector cell activating receptor upon binding. Instead, only when both the portions of the fusion protein are bound to the activating receptor on effector cells and to the antigen on target cells, the former will cross-link the activating receptor, triggering the effector cells to kill the specific antigen presenting cells. In an alternative embodiment, the Fv region activates the receptor upon binding. Standard functional assays to evaluate the target cell-killing capability by lymphocytes in the presence and absence of an Fv region or fusion protein can be set up to assess and/or screen for the ability of the Fv region to activate the receptor to which it binds.

The Fv region of the instant fusion protein can correspond to or be derived from (i.e., be a variant and/or derivative of) any suitable type of effector cell activating receptor-binding antibody. In one embodiment, the invention provides fusion proteins composed of an Fv region that corresponds to or is derived from an antibody against an activating receptor expressed on a T cell (including a NKT cell), NK cell, macrophage, dendritic cell, or neutrophil. In this respect, the invention provides fusion proteins including an Fv region derived from an antibody against a peptide presented (i.e., displayed) on an effector cell of a mammal (e.g., a human) or a functional fragment thereof. In some embodiments, the invention provides fusion proteins containing an Fv region derived from an antibody specific for a portion of a T cell receptor (TCR) or a functional variant thereof. In particular embodiments, the Fv region is specific for an invariable portion of a TCR, such as CD3 or an invariable gamma-delta TCR chain.

The sequence and composition of various TCRs and TCR subunits have been described or are known (see, e.g., GENBANK Accession Nos. AAW31109, AAW31108, AAW31107, AAW31106, AAW31105, AAW31104, and AAW31103; and U.S. Pat. No. 5,169,938) and various methods for producing antibodies against TCRs have been previously developed (including the production of antibodies against soluble TCRs or against so-called monoclonal TCRs). Such proteins can readily be used to produce antibodies, from which TCR-specific Fv regions can be derived for inclusion into a fusion protein according to the invention. Exemplary anti-TCR antibody production methods, antibodies, and related principles are described in, e.g., Necker, et al. (1991) Eur. J. Immunol. 21 (12):3035-40; Brodnicki, et al. (1996) Mol. Immunol. 33 (3):253-63; Tsang, et al. (2005) Vet. Immunol. Immunopathol. 103 (1-2):113-127; Pavlistova, et al. (2003) Immunol. Lett. 88 (2):105-8; Kubo, et al. (1989) J. Immunol. 142 (8):2736-42; U.S. Pat. Nos. 5,616,472; 5,766,947; 5,980,892; and 6,392,020. Antibodies against TCRs also are currently commercially available. Examples of commercially available anti-TCR Abs include Serotec catalog numbers (MCA987; MCA987T; MCA990; MCA990T; MCA990F; MCA990FT (Serotec, Varilhes, France).

As indicated herein, one embodiment of the invention embraces fusion proteins containing an Fv region that is specific for CD3. Anti-CD3 antibodies, anti-CD3 antibody fragments, derivatives of such proteins, and principles related to the production and use of such antibodies are known (see, e.g., Dunstone, et al. (2004) Acta Crystallogr. D Biol. Crystallogr. 60 (Pt 8):1425-8; Le Gall, et al. (2004) J. Immunol. Methods 285 (1):111-27; Renders, et al. (2003) Clin. Exp. Immunol. 133(3):307-9; Norman, et al. (2000) Transplantation 70 (12):1707-12; Cole, et al. (1997) J. Immunol. 159 (7):3613-21; Arakawa, et al. (1996) J. Biochem. (Tokyo) 120 (3):657-62; Adair, et al. (1994) Hum. Antibodies Hybridomas 5 (1-2):41-7; U.S. Patent Application Nos. 20040202657, 20040175786, 20040058445, and 20030216551, International Patent Application WO 91/09968, and U.S. Pat. Nos. 6,890,753; 6,750,325; 6,706,265; 6,406,696; 6,143,297; 6,113,901; 5,968,509; 5,929,212; 5,834,597; 5,658,741; 5,585,097; and 5,527,713. An example of a commercially available anti-CD3 antibody is the murine OKT3 antibody. Light chain and heavy chain variable sequences from OKT3 are available under GENBANK Accession No. BAA11539. Such sequences, or highly similar sequences that retain specificity for a target CD3, can form, in whole or in part, an Fv region in a fusion protein according to present invention. In particular embodiments, the Fv region of the instant fusion protein contains CD3-specific heavy chain CDRs of the sequences (a) Ser-Phe-Pro-Met-Ala (SEQ ID NO:1), (b) Thr-Ile-Ser-Thr-Ser-Gly-Gly-Arg-Thr-Tyr-Tyr-Arg-Asp-Ser-Val-Lys-Gly (SEQ ID NO:2), and (c) Phe-Arg-Gln-Tyr-Ser-Gly-Gly-Phe-Asp-Tyr (SEQ ID NO:3) and/or light chain CDRs of the sequences (d) Thr-Leu-Ser-Ser-Gly-Asn-Ile-Glu-Asn-Asn-Tyr-Val-His (SEQ ID NO:4), (e) Asp-Asp-Asp-Lys-Arg-Pro-Asp (SEQ ID NO:5), and (f) His-Ser-Tyr-Val-Ser-Ser-Phe-Asn-Val (SEQ ID NO:6).

Additional anti-CD3 antibody sequences, portions of which may be directly used as Fv regions that bind effector cell activating receptors, or that may be modified to produce functional variants for inclusion in fusion protein of this invention, are known under GENBANK Accession Nos. AAC28461 and AAC28462 (related light chain and heavy chain precursors, respectively); AAA39159 and AAA39272 (related light chain and heavy chain variable sequences, respectively); AAB81028 and AAB81027 (related heavy chain and light chain variable sequences); CAB63951; CAC10847; AAC62751; AAC28464; AAB81026; AAB81025; and CAB65246; and Leo, et al. (1987) Proc. Natl. Acad. Sci. USA 84 (5):1374-1378; Bruenke, et al. (2004) Br. J. Haematol. 125(2):167-79.

In another embodiment, the invention provides fusion proteins containing Fv regions that are specific for CD16. As with other specific and exemplary sequences provided herein, variants of the particular CD3-specific and CD16-specific sequences also or alternatively may be in fusion proteins of the invention. Moreover, the invention provides fusion proteins including Fv regions that corresponds to at least a portion of an antibody against a natural killer T (NKT) cell surface protein or a functional variant of such an antibody. Natural Killer T cells (NKT cells) are a unique subset of lymphocytes that express natural killer (NK) and T cell receptors (TCR). NKT cells generally display αβ TCRs and commonly one or more NK cell receptors. NKT cells can be characterized by the presence of various cell surface molecules (various proposals for subsets of NKT cells have been made; see, e.g., Kronenberg, et al. (2002) Nat. Rev. Immunol. 2:557-568; Godfrey, et al. (2004) Nat. Rev. Immunol. 4:231-237), such as NK1.1 or NKR-P1A (CD161) and a TCR. Many NKT cells can be characterized as containing a limited repertoire of TCRs (Vα14/Jα18 paired with Vβ8.2, Vβ7 or Vβ2). Thus, fusion proteins targeting a large set of NKTs can be obtained by inclusion of an Fv region derived from an antibody that binds to such TCRs. The sequences of several NKT receptors are known (see, e.g., Lanier, et al. (1994) J. Immunol. 153 (6):2417-2428 and GENBANK Accession No. 138700), such that antibodies against NKT cell receptors can readily be obtained using known methods. Examples of NKT cell receptor-specific antibodies are known in the art (see, e.g., Maruoka, et al. (1998) Biochem. Biophys. Res. Commun. 242 (2):413-8).

According to particular embodiments, the fusion protein of the invention contains an Fv region derived from an antibody against CD3, CD4, CD8, CD16, CD28, CD16, NKp30, NKp44, or NKp46. In some embodiments, the Fv region is not operably linked to its cognate antigen.

In addition to an Fv region specific for an effector cell, the fusion protein of this invention contains at least a portion of an NK cell receptor. In particular embodiments, the fusion protein contains the functional portion of an extracellular domain of an NK cell receptor that is able to impart receptor binding. The receptor binding portion of an extracellular domain may be known or determined by standard techniques. A portion of an NK cell receptor need not be limited to the extracellular domain of the membrane protein. Thus, transmembrane and/or intracellular sequences of such a protein may be included in a fusion protein of the invention where the presence of such sequences does not deter from the functionality of the fusion protein.

In certain embodiments, the portion of the NK cell receptor is characterized as being presented on or expressed by cells associated with a disease state normally regulated by effector lymphocytes, e.g., cancer, viral infection, or the like. Thus, for example, a typical NK cell receptor may correspond to a functional portion of a receptor for cell stress-associated molecules, such as a MIC molecule (e.g., MIC-A or MIC-B) or a ULBP (e.g., Rae-1, Mult-1, H-60, ULBP2, ULBP3, ULBP4, HCMV UL18, or Rae-1β) or a pathogen-associated molecule such as a viral hemagglutinin.

Such NK cell receptors may be, e.g., an immunoglobulin super family (IgSF) receptor. An NK cell receptor may be a natural cytotoxicity receptor (NCR). A NK cell receptor alternatively also may be an activating KIR. The structures of a number of NK cell receptors have been elucidated. To better illustrate the invention, types of well-understood NK cell receptors with reference to particular examples thereof, are described herein. However, several additional NK cell receptors are known besides those receptors explicitly described herein (see, e.g., Farag, et al. (2003) Expert Opin. Biol. Ther. 3 (2):237-250).

NK cell receptors can be divided into activating and inhibitory receptors. Many NK cell activating receptors belong to the Ig superfamily (IgSF) (such receptors also are referred to as Ig-like receptors). Activating Ig-like NK receptors include, e.g., CD2, CD16, CD69, DNAX accessory molecule-1 (DNAM-1), 2B4, NK1.1; activating killer immunoglobulin (Ig)-like receptors (KIRs); ILTs/LIRs; and natural cytotoxicity receptors (NCRs) such as NKp44, NKp46, and NKp30. Several other NK cell activating receptors belong to the CLTR superfamily (e.g., NKRP-1, CD69; CD94/NKG2C and CD94/NKG2E heterodimers, NKG2D homodimer, and in mice, activating isoforms of Ly49 (such as Ly49A-D)). Still other NK cell activating receptors (e.g., LFA-1 and VLA-4) belong to the integrin protein superfamily and other activating receptors may have even other distinguishable structures. Many NK cell activating receptors possess extracellular domains that bind to MHC-I molecules, and cytoplasmic domains that are relatively short and lack the inhibitory (ITIM) signaling motifs characteristic of inhibitory NK receptors. The transmembrane domains of these receptors typically include a charged amino acid residue that facilitates their association with signal transduction-associated molecules such as CD3ζ, FcεRIγ, DAP12, and DAP10 (2B4, for example, appears to be an exception to this general rule), which contain short amino acid sequences termed an “immunoreceptor tyrosine-based activating motif” (ITAMs) that propagate NK cell-activating signals. Receptor 2B4 contains four so-called ITSM motifs (Immunoreceptor Tyrosine-based Switch Motifs) in its cytoplasmic tail; ITSM motifs can also be found in the NK cell activating receptors CS1/CRACC and NTB-A.

Specific examples of activating NK cell receptors of use in the fusion protein of this invention include, but are not limited to, 2B4; NKR-P1A; NKR-P1B; NKR-P1C; NKG2C; NKG2D; NKG2E; CD16, CD244, CD69; FcεRIII; activating KIRs such as p50.1 (KIR2DS1), p50.2, and p50.3; natural cytotoxicity receptors (NCRs) such as NKp46, NKp30, and NKp44; activating Ly49 molecules (e.g., Ly49D, Ly49H); and ILTs/LIRs.

Activating isoforms of human KIRs (e.g., KIR2DS and KIR3DS) and murine Ly-49 proteins (e.g., Ly-49D and Ly-49H) are expressed by some NK cells. These activating KIR receptors differ from their inhibitory counterparts by lacking inhibitory motifs (ITIMs) in their relatively shorter cytoplasmic domains and possessing a charged transmembrane region that associates with signal-transducing polypeptides, such as disulfide-linked dimers of DAP12. The most common Caucasian human haplotype, the “A” haplotype (frequency of ˜47-59%), contains only one activating KIR gene (KIR2DS4). The remaining “B” haplotypes are very diverse and contain 2-5 activating KIR loci (including KIR2DS1, -2DS2, -2DS3, and 2DS5). Fusion proteins containing one or more of each of these types of KIRs (and/or one or more of these types of KIRs in combination with KIR2DS4) are further features of the invention. In a particular embodiment, the invention provides fusion proteins containing KIR2DS4, KIR2DS3, or portions thereof.

Activating KIRs have been characterized (see, e.g., GENBANK Accession Nos. NP_{—}036446, NP_{—}839942, P43632, AAR16203, AAR16204, AAR26325, CAD10378, CAD10379, CAF05810, and CAF05811, with respect to KIR2DS4 proteins; Q14954, NP_{—}055327, AAP33625, and AAB95319, with respect to KIR2DS1 proteins; NP_{—}055034, NP_{—}036444, NP_{—}937758, NP_{—}003323, CAC40718, CAC40717, P43631, AAR16202, AAR16201, with respect to KIR2DS2 proteins; NP_{—}036445 and AAB95320, with respect to KIR2DS3 proteins; and NP_{—}055328 and Q14953, with respect to KIR2DS5 proteins (other examples also are known)).

In another embodiment, the invention provides fusion proteins containing an activating non-KIR NK cell receptor (NKCR), such as a natural cytotoxicity receptor (NCR) or, for example, NKG2D. Other examples of such targets include NKG2C/CD94, and NKRP1. These and related proteins are known in the art and can be obtained using conventional recombinant techniques. Reference can be made, in this respect, to, e.g., GENBANK Accession Nos. NP_{—}031386 and NP_{—}031386 (with respect to NKG2D proteins); CAA04922, AAG26338, and Q9GME8 (with respect to NKG2C proteins); BAB91332, CAA74663, Q9MZK9, Q9MZ41, AAC50291, CAA03845, BAA24451, and Q13241 (with respect to CD94 proteins).

In particular embodiments, the invention provides fusion proteins containing a NK cell receptor or a functional portion of a NK cell receptor selected from NKG2D, NKp46, NKp44, NKp30, NKp80, CD94, DNAM-1, or a functional variant thereof.

In one particular embodiment, the NK cell receptor of the instant fusion protein is a functional portion of NKG2D having or consisting essentially of the sequence:

(SEQ ID NO: 7)
FNQEVQIPLTESYCGPCPKNWICYKNNCYQFFDESKNWYESQASCMSQNA
SLLKVYSKEDQDLLKLVKSYHWMGLVHIPTNGSWQWEDGSILSPNLLTII
EMQKGDCALYASSFKGYIENCSTPNTYICMQRTV,

the sequence of which corresponds to the extracellular domain of NKG2D (see, Ho, et al. (1998) Proc. Natl. Acad. Sci. USA 95:6320-6325; Pende, et al., J. Exp. Med. 190 (10), 1505-1516 (1999).

In another particular embodiment, the NK cell receptor of the instant fusion protein is a functional portion of NKp44 having or consisting essentially of the sequence:

(SEQ ID NO: 8)
QSKAQVLQSVAGQTLTVRCQYPPTGSLYEKKGWCKEASALVCIRLVTSSK
PRTMAWTSRFTIWDDPDAGFFTVTMTDLREEDSGHYWCRIYRPSDNSVSK
SVRFYLVVSPASASTQTSWTPRDLVSSQTQTQSCVPPTAGARQAPESPST
IPVPSQPQNSTLRPGPAAPIA,

the sequence of which corresponds to the extracellular domain of NKp44.

In another particular embodiment, the NK cell receptor of the instant fusion protein is a functional portion of CD94 having or consisting essentially of the sequence:

(SEQ ID NO: 9)
KNSFTKLSIEPAFTPGPNIELQKDSDCCSCQEKWVGYRCNCYFISSEQKT
WNESRHLCASQKSSLLQLQNTDELDFMSSSQQFYWIGLSYSEEHTAWLWE
NGSALSQYLFPSFETFNTKNCIAYNPNGNALDESCEDKNRYICKQQLI,

the sequence of which corresponds to the extracellular domain of CD94.

NK receptors bind to a variety of different ligands on tumor cells. Accordingly, the use of different NK cell receptors will facilitate targeting of effector cells to different types of tumor cells.

Functional variants of sequences discussed herein can also be used as components of the inventive fusion protein. A “functional variant” of an Fv region or portion of an NK cell receptor refers to a protein, sequence, or portion that differs from a reference protein, sequence, or portion by one or more amino acid residue substitutions, additions, insertions, and/or deletions, but which at least substantially retains some (and desirably most or even all) of the functional attributes of the protein (in the case of antibody sequences the relevant functional attribute typically is binding to the same target with an affinity that is sufficient for the desired purpose). A variant is significantly similar in terms of sequence identity with (e.g., exhibits at least about 40%, typically at least about 50%, more typically at least about 60%, even more typically at least about 70%, commonly at least about 80%, frequently as at least about 85%, such as at least about 90%, 95%, or more identity) and usually in possession of other similar physiochemical properties to at least one (referenced) protein or amino acid sequence (which may be referred to as the “parent,” which typically is a naturally occurring (“wild-type”) molecule or molecule component).

Typically, amino acid sequence variations, such as conservative substitution variations, desirably do not substantially change the structural characteristics of the parent sequence (e.g., a replacement amino acid should not tend to disrupt secondary structure that characterizes the function of the parent sequence). Examples of art-recognized polypeptide secondary and tertiary structures are described in, e.g., Proteins, Structures and Molecular Principles, Creighton, Ed., W.H. Freeman and Company, New York (1984); Introduction to Protein Structure, Branden & Tooze, eds., Garland Publishing, New York, N.Y. (1991); and Thornton, et al. (1991) Nature 354:105. Additional principles relevant to the design and construction of peptide variants is discussed in, e.g., Collinet, et al. (2000) J. Biol. Chem. 275 (23):17428-33. Protein structure can be assessed by any number of suitable techniques, such as nuclear magnetic resonance (NMR) spectroscopic structure determination techniques, which are well-known in the art (See, e.g., Wuthrich, NMR of Proteins and Nucleic Acids, Wiley, N.Y. (1986); Wuthrich (1989) Science 243:45-50; Clore, et al. (1989) Crit. Rev. Biochem. Mol. Biol. 24:479-564; Cooke, et al. (1988) Bioassays 8:52-56), typically in combination with computer modeling methods (e.g., by use of programs such as MACROMODEL, INSIGHT, and DISCOVER), to obtain spatial and orientation requirements for structural analogs. Using information obtained by these and other suitable known techniques, structural analogs can be designed and produced through rationally-based amino acid substitutions, insertions, and/or deletions. It also is possible and often desirable that such structural information be used in concert with parent antibody sequence information to design useful antibody variants.

Advantageous sequence changes with respect to a parent sequence that frequently are sought in the production of variants are those that (1) reduce susceptibility to proteolysis, (2) reduce susceptibility to oxidation, (3) alter binding affinity of the variant sequence (typically desirably increasing affinity), and/or (4) confer or modify other physicochemical or functional properties on the associated variant/analog peptide. The skilled artisan will be aware of these and other factors in the design, production, and selection of variants In the context of antibody CDR variants, for example, it is typically desired that residues required to support and/or orientate the CDR structural loop structure(s) are retained; that residues which fall within about 10 angstroms of a CDR structural loop are unmodified or modified only by conservative amino acid residue substitutions; and/or that the sequence is subject to only a limited number of insertions and/or deletions (if any), such that CDR structural loop-like structures are retained in the variant (a description of related techniques and relevant principles is provided in, e.g., Schiweck, et al. (1997) J. Mol. Biol. 268 (5):934-51; Morea (1997) Biophys. Chem. 68 (1-3):9-16; Shirai, et al. (1996) FEBS Lett. 399 (1-2):1-8; Shirai, et al. (1999) FEBS Lett. 455 (1-2):188-97; Reckzo, et al. (1995) Protein Eng. 8 (4):389-95; and Eigenbrot, et al. (1993) J. Mol. Biol. 229 (4):969-95).

In the design, construction, and/or evaluation of CDR variants, attention typically is paid to the fact that CDR regions can vary to enable a better binding to the epitope. Antibody CDRs typically operate by building a “pocket,” or other paratope structure, into which the epitope fits. If the epitope is not fitting tightly, the antibody may not offer the best affinity. However, as with epitopes, there often are a few key residues in a paratope structure that account for most of this binding. Thus, CDR sequences can vary in length and composition significantly between antibodies for the same peptide. The skilled artisan will recognize that certain residues, such as tyrosine residues (e.g., in the context of CDR-H3 sequences), that are often significant contributors to such epitope binding, are typically desirably retained in a CDR variant.

Typically, a variant Fv region will contain less than about 10, such as less than about 5, such as 3 or less amino acid variations (differences by way of the above-described methods, e.g., substitution), in either the VH or VL regions of the Fv region with respect to a parent Fv region.

Variants of Fv region can be generated by any one or combination of techniques known in the art. For example, to improve the quality and/or diversity of antibodies against a target, the VL and VH segments of VL/VH pair(s) (or portions thereof) can be randomly mutated, typically at least within the CDR3 region of VH and/or VL, in a process analogous to the in vivo somatic mutation process responsible for affinity maturation of antibodies during a natural immune response. Such in vitro affinity maturation can be accomplished by, e.g., amplifying VH and VL regions using PCR primers complimentary to VH CDR3 or VL CDR3 encoding sequences, respectively, which primers typically are “spiked” with a random mixture of the four nucleotide bases at certain positions, such that the resultant PCR products encode VH and VL segments into which random mutations have been introduced thereby resulting (at least in some cases) in the introduction of sequence variations in the VH and/or VL CDR3 regions. Such randomly mutated VH and VL segments can thereafter be re-screened by phage display or other suitable technique for binding to target molecule(s) and advantageous variants analyzed and used to prepare functional variant sequences. Following screening, a nucleic acid encoding a selected antibody, where appropriate, can be recovered from a display package (e.g., from a phage genome) and subcloned into an appropriate vector by standard recombinant techniques. If desired, such an antibody-encoding nucleic acid can be further manipulated to create other antibody forms. To express a recombinant human antibody isolated by screening of a combinatorial library, typically a nucleic acid containing a sequence encoding the antibody is cloned into a recombinant expression vector and introduced into appropriate host cells (mammalian cells, yeast cells, etc.) under conditions suitable for expression of the nucleic acid and production of the antibody.

A convenient method for generating substitution variants is affinity maturation using phage according to methods known in the art. In order to identify candidate hypervariable region sites for modification, alanine scanning mutagenesis also can be performed to identify hypervariable region residues contributing significantly to antigen binding. Alternatively or additionally, it may be beneficial to analyze a crystal structure of the antigen-antibody complex to identify contact points between the antibody and antigen. Such contact residues and neighboring residues are likely suitable candidates for substitution. Useful methods for rational design of CDR sequence variants are described in, e.g., WO91/09967 and WO93/16184.

Other methods for generating CDR variants include the removal of nonessential residues (see, Studnicka, et al. (1994) Protein Engineering 7:805-814), CDR walking mutagenesis and other artificial affinity maturation techniques (see, e.g., Yang, et al. (1995) J. Mol. Biol. 254 (3):392-403), and CDR shuffling techniques.

As indicated, the basic properties of “parent” sequences that are desirably retained in variant sequences are similar specificity and suitable affinity for target molecules bound by the parent (retention of at least a substantial proportion of the affinity of the parent sequence for its target, e.g., CD3 in the case of an anti-CD3 antibody). Typically, a suitable affinity for a target falls in the range of about 10⁴ to about 10¹⁰ M⁻¹ (e.g., about 10⁷ to about 10⁹ M⁻¹). A variant Fv region, for example, may have an average disassociation constant (KD) of about 7×10⁻⁹ M or more with respect to a target (e.g., an activating NK cell receptor), as determined by, e.g., surface plasmon resonance (SPR) screening (such as by analysis with a BIACORE SPR analytical device). Typically, variant sequence antibody portions also or alternatively can be characterized by exhibiting target binding with a disassociation constant of less than about 100 nM, less than about 50 nM, less than about 10 nM, about 5 nM or less, about 1 nM or less, about 0.5 nM or less, about 0.1 nM or less, about 0.01 nM or less, or even about 0.001 nM or less.

Fusion proteins as described herein can be produced using routine genetic engineering. This typically involves appending the cDNA sequence of the two proteins of interest in-frame through ligation or overlap extension PCR. The resulting chimeric DNA molecule is then inserted into an expression vector and expressed by a recombinant host cell (e.g., a bacterial, yeast, mammalian, or insect cell) to yield the fusion protein. The production of recombinant proteins in this manner is routinely practiced in the art and any conventional or commercially available expression system can be employed.

In some embodiments, the Fv region and NK receptor molecule are separated by a linker (or “spacer”) peptide. Such spacers are well-known in the art (e.g., polyglycine) and typically allow for proper folding of one or both of the components of the fusion protein. In some embodiments, the fusion protein of the invention further contains a tag for identification and purification of the fusion protein. Such tags are well-known in the art and include, but are not limited to, GST protein, FLAG peptide, or a hexa-his peptide (aka, a 6xhis-tag), which can be isolated using nickel or cobalt resins (affinity chromatography).

In so far as the instant fusion protein finds application in the treatment and prevention of disease, another feature of the invention relates to compositions that include fusion proteins of the invention, such as pharmaceutical compositions containing an effective amount of a fusion protein of the invention (such as a therapeutically effective amount (therapeutic dose) of such a fusion protein). Compositions containing a fusion protein of the invention that are intended for pharmaceutical use typically contain at least a physiologically effective amount of the fusion protein, and commonly desirably contain a therapeutically effective amount of a fusion protein, or a combination of a fusion protein and additional active/therapeutic agents (combination therapies and compositions are discussed elsewhere herein).

A “therapeutically effective amount” refers to an amount of a biologically active compound or composition that, when delivered in appropriate dosages and for appropriate periods of time to a host that typically is responsive for the compound or composition, is sufficient to achieve a desired therapeutic result in a host and/or typically able to achieve such a therapeutic result in substantially similar hosts (e.g., patients having similar characteristics as a patient to be treated). A therapeutically effective amount of a fusion protein may vary according to factors such as the disease state, age, sex, and weight of the individual, and the ability of the fusion protein to elicit a desired response in the individual. A therapeutically effective amount is also one in which any toxic or detrimental effects of the Fv region are outweighed by the therapeutically beneficial effects. Exemplary therapeutic effects include, e.g., a reduction in the severity of a disease, disorder, or related condition in a particular subject or a population of substantial similar subject; a reduction in one or more symptoms or physiological conditions associated with a disease, disorder, or condition; or a prophylactic effect. A reduction of the severity of a disease can include, for example, a measurable reduction in the spread of a disorder (e.g., the spread of a cancer in a patient); an increase in the chance of a positive outcome in a subject (e.g., an increase of at least about 5%, 10%, 15%, 20%, 25%, or more); an increased chance of survival or lifespan; and/or a measurable reduction in one or more biomarkers associated with the presence of the disease state (e.g., a reduction in the amount and/or size of tumors in the context of cancer treatment; a reduction in viral load in the context of virus infection treatment; etc.). A therapeutically effective amount can be measured in the context of an individual subject or, more commonly, in the context of a population of substantial similar subjects (e.g., a number of human patients with a similar disorder enrolled in a clinical trial involving a fusion protein composition or a number of non-human mammals having a similar set of characteristics being used to test a fusion protein in the context of preclinical experiments).

A “prophylactically effective amount” refers to an amount of an active compound or composition that is effective, at dosages and for periods of time necessary, in a host typically responsive to such compound or composition, to achieve a desired prophylactic result in a host or typically able to achieve such results in substantially similar hosts. Exemplary prophylactic effects include a reduction in the likelihood of developing a disorder, a reduction in the intensity or spread of a disorder, an increase in the likelihood of survival during an imminent disorder, a delay in the onset of a disease condition, a decrease in the spread of an imminent condition as compared to in similar patients not receiving the prophylactic regimen, etc. Typically, because a prophylactic dose is used in subjects prior to or at an earlier stage of disease, the prophylactically effective amount will be less than the therapeutically effective amount for a particular fusion protein. A prophylactic effect also can include, e.g., a prevention of the onset, a delay in the time to onset, a reduction in the consequent severity of the disease as compared to a substantially similar subject not receiving fusion protein composition, etc.

A “physiologically effective” amount is an amount of an active agent that upon administration to a host that is normally responsive to such an agent results in the induction, promotion, and/or enhancement of at least one physiological effect associated with modulation of effector lymphocyte activity (e.g., increase in NK cell-associated apoptosis; increase in NK cell-associated IFNγ secretion; etc.). A therapeutically effective amount typically also is prophylactically effective and physiologically effective, but the reverse is typically not true (i.e., a physiologically effective amount may be too low of an amount or too high of an amount to be therapeutically effective).

Terms such as “treat”, “treating”, and “treatment” herein refer to the delivery of an effective amount of a therapeutically active compound or composition, such as a fusion protein composition of the invention, with the purpose of preventing any symptoms or disease state to develop or with the purpose of easing, ameliorating, or eradicating (curing) such symptoms or disease states already developed. The term “treatment” is thus meant to include prophylactic treatment. However, it will be understood that therapeutic regimens and prophylactic regimens of the invention also can be considered separate and independent aspects of this invention.

A fusion protein can be combined with one or more pharmaceutically acceptable carriers (diluents, excipients, and the like) and/or adjuvants appropriate for one or more intended routes of administration to provide compositions that are pharmaceutically acceptable. Pharmaceutically acceptable compositions comprising a therapeutic dose of a fusion protein of the invention may be referred to as “pharmaceutical compositions”. Acceptability of a composition and its components is generally made in terms of toxicity, adverse side effects, undesirable immunogenicity, etc., as will be readily determinable by standard methods.

Pharmaceutically acceptable carriers generally include any and all suitable solvents, dispersion media, coatings, antibacterial and antifungal agents, isotonic and absorption delaying agents, and the like that are physiologically compatible with a fusion protein. Examples of pharmaceutically acceptable carriers include water, saline, phosphate buffered saline, dextrose, glycerol, ethanol, and the like, as well as combinations of any thereof. In many cases, it can be desirable to include isotonic agents, for example, sugars, polyalcohols such as mannitol, sorbitol, or sodium chloride in such a composition. Pharmaceutically acceptable substances such as wetting agents or minor amounts of auxiliary substances such as wetting agents or emulsifying agents, preservatives or buffers, which desirably can enhance the shelf life or effectiveness of the fusion protein, related composition, or combination.

Fusion protein compositions, related compositions (e.g., compositions containing nucleic acids encoding one of the inventive fusion proteins), and combinations according to the invention may be in a variety of suitable forms. Such forms include, for example, liquid, semi-solid and solid dosage forms, such as liquid solutions (e.g., injectable and infusible solutions), dispersions or suspensions, emulsions, microemulsions, tablets, pills, powders, liposomes, dendrimers and other nanoparticles (see, e.g., Baek, et al. (2003) Methods Enzymol. 362:240-9; Nigavekar, et al. (2004) Pharm Res. 21 (3):476-83), microparticles, and suppositories. The optimal form for any fusion protein-associated composition depends on the intended mode of administration, the nature of the composition or combination, and therapeutic application or other intended use. Formulations also can include, for example, powders, pastes, ointments, jellies, waxes, oils, lipids, lipid (cationic or anionic) containing vesicles, DNA conjugates, anhydrous absorption pastes, oil-in-water and water-in-oil emulsions, emulsions, carbowax (polyethylene glycols of various molecular weights), semi-solid gels, and semi-solid mixtures containing carbowax. Any of the foregoing mixtures may be appropriate in treatments and therapies in accordance with the present invention, provided that the binding of the fusion protein to its targets is not significantly inhibited by the formulation and the formulation is physiologically compatible and tolerable with the route of administration. See also, e.g., Powell, et al. (1998) PDA J. Pharm. Sci. Technol. 52:238-311 and the citations therein for additional information related to excipients and carriers well-known to pharmaceutical chemists. In some embodiments, fusion proteins are administered in liposomes (immunoliposomes). The production of liposomes is well-known in the art. Immunoliposomes also can be targeted to particular cells by standard techniques.

Furthermore, wherein the fusion protein is delivered in the form of a nucleic acid molecule encoding the same, the said nucleic acid molecule can be administered via a viral vector. Viral vectors, such as recombinant adenovirus, adenovirus-associated virus (AAV), Herpes simplex virus (HSV) can be used in localized in vivo production of the instant fusion protein in a subject in need of treatment. Bacteria harboring DNA for the instant fusion protein can also be used to produce the fusion protein.

Moreover, the instant fusion protein can be delivered to a subject via cell vehicles. Myeloid cells, such as macrophages or dendritic cells have a strong capacity to infiltrate tumors (especially solid tumors). In a “Trojan horse” approach, myeloid cells can be genetically modified to express the instant fusion protein to deliver the same to tumor tissue. In this approach, locally expressed fusion protein would be expected to engage both infiltrated T cells and tumor cells, leading to tumor destruction.

Typically, compositions in the form of injectable or infusible solutions, such as compositions similar to those used for passive immunization of humans with other antibodies, are used for delivery of fusion proteins of the invention. A typical mode for delivery of fusion protein compositions is by parenteral administration (e.g., intravenous, subcutaneous, intraperitoneal, and/or intramuscular administration). In one embodiment, a fusion protein is administered to a human patient by intravenous infusion or injection. In another aspect, a fusion protein is administered by intramuscular or subcutaneous injection. Intratumor administration also may be useful in certain therapeutic regimens. Thus, fusion proteins may, for example, be applied in a variety of solutions. Suitable solutions for use in accordance with the invention typically are sterile, dissolve sufficient amounts of the antibody and other components of the composition (e.g., an immunomodulatory cytokine such as GM-CSF, IL-2, and/or KGF), stable under conditions for manufacture and storage, and not harmful to the subject for the proposed application.

In another embodiment, compositions of the invention are formulated for oral administration, for example, with an inert diluent or an assimilable edible carrier. The fusion protein (and other ingredients, if desired to be included) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

In the case of combination compositions, fusion proteins can be coformulated with and/or coadministered with one or more additional therapeutic agents (e.g., an antigenic peptide and/or an immunostimulatory cytokine). Such combination therapies may require lower dosages of the fusion protein and/or the co-administered agents, thus avoiding possible toxicities or complications associated with the various monotherapies. There are a number of agents that may be advantageously combined with fusion proteins of the invention and the selection of such agents will depend on the intended use of the fusion protein, the components of the fusion protein, etc. For example, the present invention embraces combination therapies that include a fusion protein of the invention that is capable of inducing or promoting a response against a cancerous or pre-cancerous condition and at least one second anti-cancer agent. Accordingly, in particular embodiments, the instant fusion protein is used as an adjuvant therapy in the treatment of cancer. As another example, the invention embraces combination therapies that include a fusion protein of the invention that is capable of inducing or promoting a therapeutic response against a viral infection and at least one second anti-viral agent.

In the case of compositions and methods used to treat cancer or as prophylaxis against cancer in the case of a patient at risk of developing a cancer (e.g., a patient in a period of remission, a patient having a detected precancerous condition, etc.), fusion proteins of the invention may be combined with one or more anti-cancer second agents in a method for enhancing immunity against the tumor. Such secondary agents can be any suitable antineoplastic therapeutic agent, such as an antineoplastic immunogenic peptide, antibody, or small molecule drug. Drugs employed in cancer therapy may have a cytotoxic or cytostatic effect on cancer cells, or may reduce proliferation of the malignant cells. Among the texts providing guidance for cancer therapy is Cancer, Principles and Practice of Oncology, 4th Edition, DeVita et al., Eds. J. B. Lippincott Co., Philadelphia, Pa. (1993). An appropriate therapeutic approach is chosen according to such factors as the particular type of cancer and the general condition of the patient, as is recognized in the pertinent field. Examples of anticancer agents include but are not limited to, cytotoxic agents such as Vinca alkaloid, taxanes, and topoisomerase inhibitors; antisense nucleic acids such as augmerosen/G3139, LY900003 (ISIS 3521), ISIS 2503, OGX-011 (ISIS 112989), LE-AON/LEraf-AON (liposome encapsulated c-raf antisense oligonucleotide/ISIS-5132), MG98, and other antisense nucleic acids that target PKCα, clusterin, IGFBPs, protein kinase A, cyclin D1, or Bcl-2; anticancer nucleozymes such as angiozyme (Ribozyme Pharmaceuticals); tumor suppressor-encoding nucleic acids such as a p53, BRCA1, RB1, BRCA2, DPC4 (Smad4), MSH2, MLH1, and DCC; oncolytic viruses such as oncolytic adenoviruses and herpes viruses; anti-cancer immunogens such as a cancer antigen/tumor-associated antigen, e.g., an epithelial cell adhesion molecule (Ep-CAM/TACSTD1), mucin 1 (MUC1), carcinoembryonic antigen (CEA), tumor-associated glycoprotein 72 (TAG-72), gp100, Melan-A, MART-1, KDR, RCAS1, MDA7, cancer-associated viral vaccines, tumor-derived heat shock proteins, and the like; anti-cancer cytokines, chemokines, or combination thereof; inhibitors of angiogenesis, neovascularization, and/or other vascularization; and/or any other conventional anticancer agent including fluoropyrimidiner carbamates, non-polyglutamatable thymidylate synthase inhibitors, nucleoside analogs, antifolates, topoisomerase inhibitors, polyamine analogs, mTOR inhibitors, alkylating agents, lectin inhibitors, vitamin D analogs, carbohydrate processing inhibitors, antimetabolism folate antagonists, thumidylate synthase inhibitors, antimetabolites, ribonuclease reductase inhibitors, dioxolate nucleoside analogs, and chemically modified tetracyclines.

The invention also provides kits containing one or more fusion proteins or related agents (e.g., fusion protein-encoding nucleic acids, or vectors or host cells containing the same). A kit may include, in addition to the fusion protein, other therapeutic agents. A kit may also include instructions for use in a therapeutic method. Such instructions can be, for example, provided on a device included in the kit. In another preferred embodiment, the kit includes a fusion protein, related compound, or combination composition in a highly stable form (such as in a lyophilized form) in combination with pharmaceutically acceptable carrier(s) that can be mixed with the highly stable composition to form an injectable composition for near term administration. Such kits also can be provided with one or more other non-active pharmaceutical composition ingredients, such as a stabilizer, a preservative, a solubilizer, a solvent, a solute, a flavorant, a coloring agent, etc.

The invention further embraces prophylactic and therapeutic methods involving fusion proteins, fusion protein compositions, and/or related compositions. Fusion proteins of the invention can be useful in a variety of therapeutic and prophylactic regimens including, for example, the treatment of cancer, pathogen infections, and immune system-related disorders. Accordingly, in one embodiment, the invention provides a method for preventing cancer development or progression in a mammalian host, such as a human subject, with one or more precancerous lesions or a subject predisposed to cancer, e.g., as a result of genetic mutation, family history or exposure to a carcinogenic agent. In another embodiment the invention provides a method of treating cancer in a mammalian host, such as a human subject, having a detectable level of cancer cells. In accordance with these embodiments, the subject is administered a fusion protein, a fusion protein composition, or a related composition (e.g., a nucleic acid encoding a fusion protein), in an amount sufficient to detectably reduce the development or progression of the cancer in the subject. In particular embodiments, the fusion protein desirably includes the extracellular domain of NKG2D. NKG2D binds to multiple ligands, including members of the MIC-A, MIC-B and RAET-1 protein families. These all are stress-inducible ligands whose expression is induced in several types of tumors. For instance, in most normal tissues, MIC-A is not expressed, but MIC-A is upregulated in various types of tumors, including epithelial breast, lung and colorectal cancers, leukemias, and gliomas (Groh, et al. (1999) Proc. Natl. Acad. Sci. USA 96:6879-84).

Cancer cells are cells that divide and reproduce abnormally with uncontrolled growth. Cancers are generally composed of single or several clones of cells that are capable of partially independent growth in a host (e.g., a benign tumor) or fully independent growth in a host (malignant cancer). Cancer cells arise from host cells via neoplastic transformation (i.e., carcinogenesis). Terms such as “preneoplastic,” “premalignant,” and “precancerous” with respect to the description of cells and/or tissues herein refer to cells or tissues having a genetic and/or phenotypic profile that signifies a significant potential of becoming cancerous. Usually such cells can be characterized by one or more differences from their nearest counterparts that signal the onset of cancer progression or significant risk for the start of cancer progression. Such precancerous changes, if detectable, can usually be treated with excellent results. In general, a precancerous state will be associated with the incidence of neoplasm(s) or preneoplastic lesion(s). Examples of known and likely preneoplastic tissues include ductal carcinoma in situ (DCIS) growths in breast cancer, cervical intra-epithelial neoplasia (CIN) in cervical cancer, adenomatous polyps of colon in colorectal cancers, atypical adenomatous hyperplasia in lung cancers, and actinic keratosis (AK) in skin cancers. Pre-neoplastic phenotypes and genotypes for various cancers, and methods for assessing the existence of a preneoplastic state in cells, have been characterized. See, e.g., Medina (2000) J. Mammary Gland Biol. Neoplasia 5 (4):393-407; Krishnamurthy, et al. (2002) Adv. Anat. Pathol. 9 (3):185-97; Ponten (2001) Eur. J. Cancer October 37 Suppl 8:S97-113; Niklinski, et al. (2001) Eur. J. Cancer Prev. 10 (3):213-26; Walch, et al. Pathobiology (2000) 68 (1):9-17; Busch (1998) Cancer Surv. 32:149-79. Gene expression profiles can increasingly be used to differentiate between normal, precancerous, and cancer cells. For example, familial adenomatous polyposis genes prompt close surveillance for colon cancer; mutated p53 tumor-suppressor gene flags cells that are likely to develop into aggressive cancers; osteopontin expression levels are elevated in premalignant cells, and increased telomerase activity also can be a marker of a precancerous condition (e.g., in cancers of the bladder and lung). In one aspect, the invention relates to the treatment of precancerous cells. In another aspect, the invention relates to the preparation of medicaments for treatment of precancerous cells.

In general, fusion proteins of the invention can be used to treat subjects suffering from any stage of cancer (and to prepare medicaments for reduction, delay, or other treatment of cancer). Effective treatment of cancer (and thus the reduction thereof) can be detected by any variety of suitable methods. Methods for detecting cancers and effective cancer treatment include clinical examination (symptoms can include swelling, palpable lumps, enlarged lymph nodes, bleeding, visible skin lesions, and weight loss); imaging (X-ray techniques, mammography, colonoscopy, computed tomography (CT and/or CAT) scanning, magnetic resonance imaging (MRI), etc.); immunodiagnostic assays (e.g., detection of CEA, AFP, CA125, etc.); antibody-mediated radioimaging; and analyzing cellular/tissue immunohistochemistry. Other examples of suitable techniques for assessing a cancerous state and effective cancer treatment include PCR and RT-PCR (e.g., of cancer cell associated genes or “markers”), biopsy, electron microscopy, positron emission tomography (PET), computed tomography, magnetic resonance imaging (MRI), karyotyping and other chromosomal analysis, immunoassay/immunocytochemical detection techniques (e.g., differential antibody recognition), histological and/or histopathologic assays (e.g., of cell membrane changes), cell kinetic studies and cell cycle analysis, ultrasound or other sonographic detection techniques, radiological detection techniques, flow cytometry, endoscopic visualization techniques, and physical examination techniques.

In general, delivering fusion proteins of the invention to a subject (either by direct administration or expression from a nucleic acid) according to the methods disclosed herein can be used to reduce, treat, prevent, or otherwise ameliorate any aspect of cancer in a subject. In this respect, treatment of cancer can include, e.g., any detectable decrease in the rate of normal cells transforming to neoplastic cells (or any aspect thereof), the rate of proliferation of pre-neoplastic or neoplastic cells, the number of cells exhibiting a pre-neoplastic and/or neoplastic phenotype, the physical area of a cell media (e.g., a cell culture, tissue, or organ) containing pre-neoplastic and/or neoplastic cells, the probability that normal cells and/or preneoplastic cells will transform to neoplastic cells, the probability that cancer cells will progress to the next aspect of cancer progression (e.g., a reduction in metastatic potential), or any combination thereof. Such changes can be detected using any of the above-described techniques or suitable counterparts thereof known in the art, which typically are applied at a suitable time prior to the administration of a therapeutic regimen so as to assess its effectiveness. Times and conditions for assaying whether a reduction in cancer has occurred will depend on several factors including the type of cancer, type and amount of fusion protein, related composition, or combination composition being delivered to the host. The accomplishment of these goals by delivery of fusion proteins of the invention is another advantageous facet of this invention.

The methods of the invention can be used to treat a variety of cancers. Forms of cancer that may be treated by the delivery or administration of fusion proteins, fusion protein compositions, and combination compositions provided by the invention include squamous cell carcinoma, leukemia, acute lymphocytic leukemia, acute lymphoblastic leukemia, B-cell lymphoma, T-cell lymphoma, Hodgkins lymphoma, non-Hodgkins lymphoma, hairy cell lymphoma, Burketts lymphoma, acute or chronic myelogenous leukemias, promyelocytic leukemia, fibrosarcoma, rhabdomyoscarcoma; melanoma, seminoma, teratocarcinoma, neuroblastoma, glioma, astrocytoma, neuroblastoma, glioma, schwannomas; fibrosarcoma, rhabdomyoscaroma, osteosarcoma, melanoma, xeroderma pigmentosum, keratoacanthoma, seminoma, thyroid follicular cancer, and teratocarcinoma. Fusion proteins also can be useful in the treatment of other carcinomas of the bladder, breast, colon, kidney, liver, lung, ovary, prostate, pancreas, stomach, cervix, thyroid or skin. Fusion proteins also may be useful in treatment of other hematopoietic tumors of lymphoid lineage, other hematopoietic tumors of myeloid lineage, other tumors of mesenchymal origin, other tumors of the central or peripheral nervous system, and/or other tumors of mesenchymal origin. Advantageously, the methods of the invention also may be useful in reducing cancer progression in prostate cancer cells, melanoma cells (e.g., cutaneous melanoma cells, ocular melanoma cells, and/or lymph node-associated melanoma cells), breast cancer cells, colon cancer cells, and lung cancer cells. The methods of the invention can be used to treat both tumorigenic and non-tumorigenic cancers (e.g., non-tumor-forming hematopoietic cancers). The methods of the invention are particularly useful in the treatment of epithelial cancers (e.g., carcinomas) and/or colorectal cancers, breast cancers, lung cancers, vaginal cancers, cervical cancers, and/or squamous cell carcinomas (e.g., of the head and neck). Additional potential targets include sarcomas and lymphomas. Additional advantageous targets include solid tumors and/or disseminated tumors (e.g., myeloid and lymphoid tumors, which can be acute or chronic).

In addition to cancer treatment, the present invention also features a method of treating a pathogen infection in a subject or host. This method involves administering or otherwise delivering a therapeutically effective amount of a fusion protein, a fusion protein composition, or combination composition so as to reduce the severity, spread, symptoms, or duration of such infection. Such pathogen infections include, but are not limited to diseases caused by bacteria, protozoa, fungi or viruses.

In particular embodiments, a viral infection is treated. Any virus normally associated with the activity of effector lymphocytes, such as NK cells, can be treated by the method. For example, such a method can be used to treat infection by one or more viruses selected from hepatitis type A, hepatitis type B, hepatitis type C, influenza, varicella, adenovirus, herpes simplex type I (HSV-1), herpes simplex type 2 (HSV-2), rinderpest, rhinovirus, echovirus, rotavirus, respiratory syncytial virus, papilloma virus, papilloma virus, cytomegalovirus (CMV—e.g., HCMV), echinovirus, arbovirus, huntavirus, coxsackie virus, mumps virus, measles virus, rubella virus, polio virus, and/or human immunodeficiency virus type I or type 2 (HIV-1, HIV-2). The practice of such methods may result in a reduction in the titer of virus (viral load), reduction of the number of virally infected cells, etc. In a particular embodiment, this method is practiced in immunocompromised/immunosuppressed individuals. In another embodiment, this method is practiced in subjects at relatively higher risk of immunosuppression or having a relatively defective immune system, such as in young children (e.g., children of about 10 years or less in age) or the elderly (e.g., subjects of about 65 years or more in age).

In accordance with this method of the invention, the fusion protein can be administered with or in association with anti-viral agents, such as protease inhibitor (e.g. acyclovir) in the context of HIV treatment or an anti-viral antibody (e.g., an anti-gp41 antibody in the context of HIV treatment; an anti-CD4 antibody in the context of the treatment of CMV, etc.). Numerous types of anti-viral agents for the above-described viruses are known with respect to each type of target virus.

In addition to pathogen infections, fusion proteins of the invention can be administered or otherwise delivered to a subject in association with transplantation (e.g., the grafting or insertion of cells, tissue(s) or organ(s)) to reduce undesirable host immune responses to the transplanted tissue. Similarly, fusion proteins can be administered or otherwise delivered to a subject to treat one or more disorders associated with transplant tolerance. Other applications of the instant fusion proteins include, but are not limited to the treatment of immunoproliferative diseases, immunodeficiency diseases, autoimmune diseases, inflammatory responses, and/or allergic responses.

Although the use of an anti-CD3 based “bi-specific antibody strategy” for tumor targeting has been described in the art, such antibody designs have involved anti-CD3ε mAbs linked to anti-tumor antigen mAbs (anti-CD3×anti-tumor antigen) either by fusing through routine molecular biology techniques or chemical conjugation. The instant fusion protein is novel in that a single chain Fv region is fused to an activating NK cell receptor or portion thereof. Because NK cell receptors, such as NKG2D, recognize multiple tumor cell types, this strategy can be used to treat many types of tumors. The instant fusion protein is unique in that the Fv region does not contain the Fc fragment. In this respect, non-specific binding of the instant fusion proteins to FcR-positive cells (such as macrophages, B cells, neutrophils, and dendritic cells via the Fc region) is eliminated, resulting in less non-tumor associated T cell activation and less binding and removal of the fusion protein. This design may make this fusion protein more effective than proteins with a Fc region.

### EXAMPLE 1

### Construction and Production of scFv-NKG2D

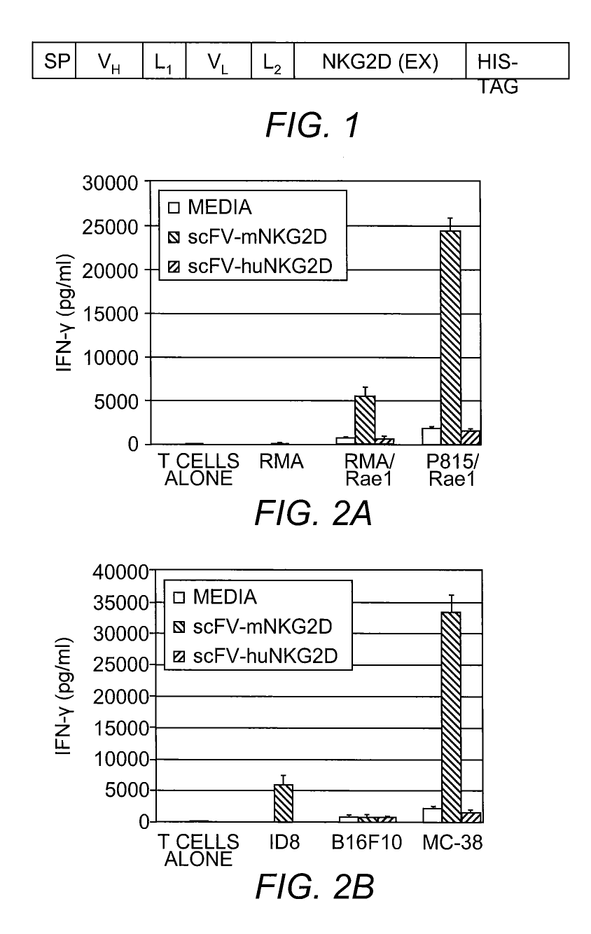

Bi-specific molecule scFv-NKG2D was generated using the anti-CD3ε binding Fv region fused to NKG2D (FIG. 1). The gene coding for the scFv portion of fusion protein scFv-NKG2D was constructed by PCR amplification of variable region of heavy chain (V_{H}) and variable region of light chain (V_{L}) using cDNA derived from an anti-mouse CD3ε hybridoma 2C11 (ATCC). V_{H} and V_{L} were linked using a flexible linker of three repeats of Gly-Gly-Gly-Gly-Ser (SEQ ID NO:10) ((G4S)₃). Signal peptide (SP) from Ig heavy chain or other type I protein (such as Dap10) was also included at the 5′ end of the recombinant DNA. The gene coding for the extracellular portion of mouse NKG2D was PCR-amplified using wild-type full-length NKG2D plasmid as template (Zhang, et al. (2005) Blood 106 (5):1544-51). Both scFv and NKG2D portions were linked in-frame with a second (G4S)₃ and cloned in a retroviral vector pFB-neo (STRATAGENE) and a mammalian expression vector pcDNA3.1 (INVITROGEN), respectively. For the convenience of protein purification, a histidine tag (6 repeats of histidine) was added at the C-terminus.

As appreciated by one skilled in the art, other scFv-NKR fusion proteins can be constructed in a similar manner. Moreover, there are other methods for making scFv fusion proteins which are known to those of skill in the art, any of which can be employed in practicing the instant invention.

### EXAMPLE 2

### Characterization of scFv-NKG2D

The activity of the scFv-NKG2D fusion protein was assessed. To demonstrate binding specificity, it was determined whether the fusion protein can bind to CD3. A T cell lymphoma cell line RMA (10⁵, CD3⁺ NKG2D⁻), which does not express ligands for NKG2D, was stained with scFv-NKG2D (0.01-1 μg/ml) followed by staining with anti-NKG2D-PE. Samples were analyzed with an Accuri C6 flow cytometer and it was shown that the scFv-NKG2D fusion protein can bind to CD3.

To demonstrate activity, it was determined whether the fusion protein could induce IFN-γ secretion. Bulk spleen cells were stimulated with ConA and IL-2 before co-culture with irradiated tumor cells. The scFv-NKG2D was subsequently added and IFN-γ amounts in the supernatants were analyzed with ELISA. The results of this analysis indicated that T cells respond to NKG2D ligand positive cells by producing IFN-γ in the presence of scFv-NKG2D (FIG. 2). These data also show that the expression of NKG2D ligands on tumor cells is required for induction of IFN-γ production.

### EXAMPLE 3

### In Vitro Tumor Killing Activity of scFv-NKG2D

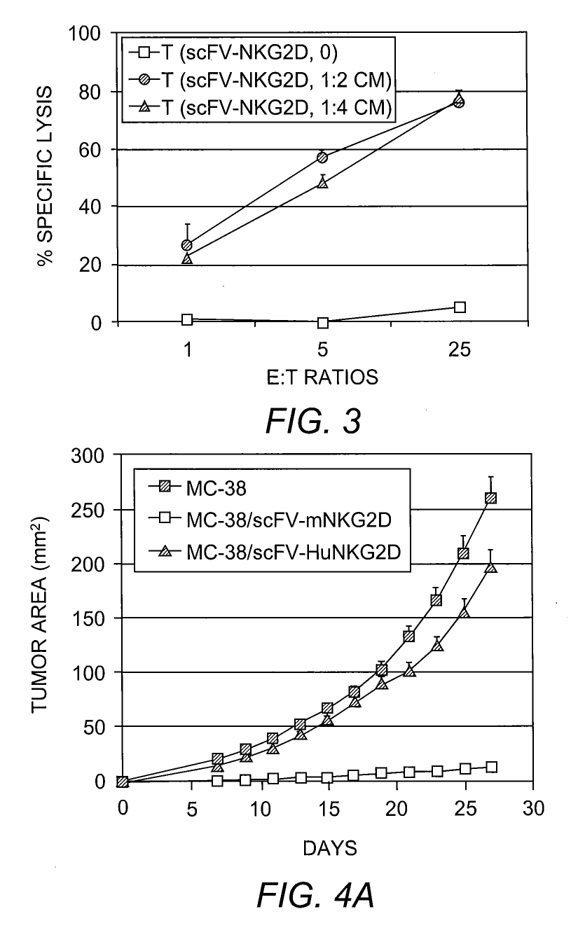

In addition to IFN-γ secretion, it was determined whether the scFv-NKG2D fusion protein could mediate tumor killing. ConA-stimulated T cells were co-cultured with NKG2D ligand-positive P815/Rae1 in the presence or absence of scFv-NKG2D and specific lysis was determined. This analysis indicated that T cells can kill NKG2D ligand-positive tumor cells in the presence of scFv-NKG2D (FIG. 3).

### EXAMPLE 4

### In Vivo Tumor Killing Activity of scFv-NKG2D

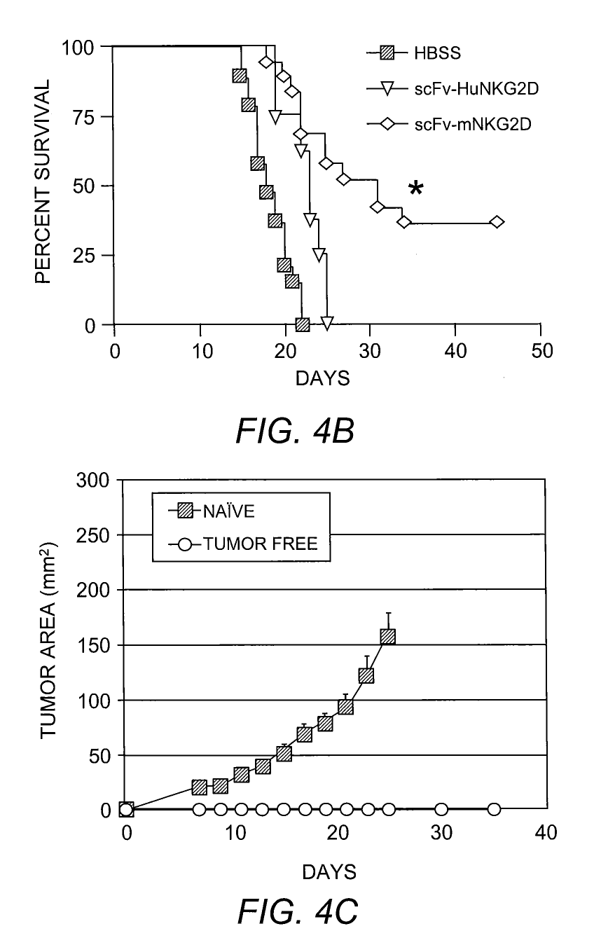

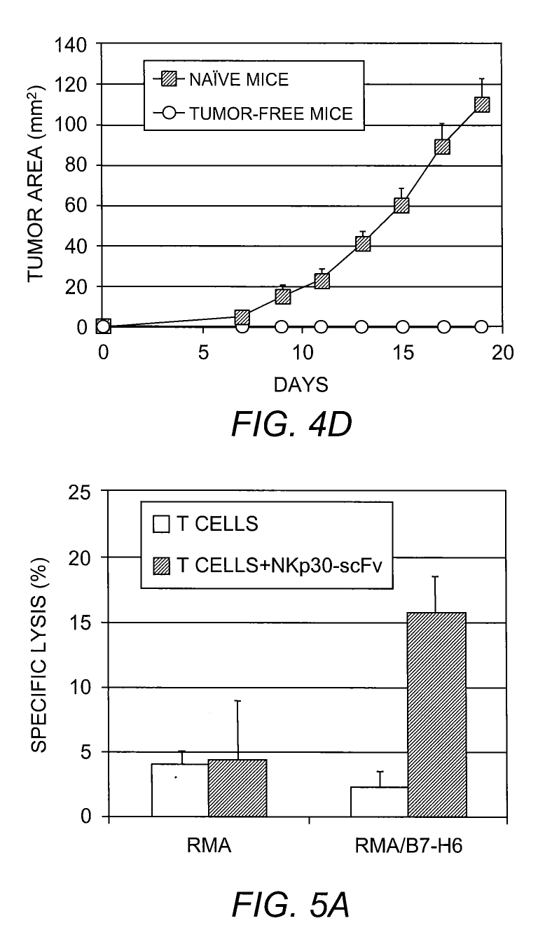

Effects on tumor growth were also analyzed. Mouse colon cancer MC-38 cells were genetically modified with a retroviral vector containing the scFv-NKG2D gene. These cells were injected s.c. into right flanks of B6 mice and tumor development was monitored. Only 3 of 10 mice developed small tumors, whereas in control groups in which wild-type MC-38 cells were given, all mice developed tumors (FIG. 4A). To demonstrate specificity, a human-NKG2D-scFv construct was also prepared and expressed by MC-38 cells as a control for the murine-NKG2D-scFv. As shown in FIG. 4A, expression of murine-NKG2D-scFv in MC-38 cells reduced or abrogated tumor growth. FIG. 4C shows that rechallenge of surviving mice from FIG. 4A also led to resistance against tumor growth in the MC-38 tumor system.

The data presented in FIG. 4B show treatment with purified NKG2D-scFv protein on days 5, 7, and 9 after lymphoma (RMA-RG tumor) injection. This treatment resulted in 40% long-term survivors. In addition, these surviving mice were resistant to tumor rechallenge (FIG. 4D), thus showing the induction of immunity against the tumor by this treatment.

These data demonstrate that the exemplary monomeric bi-functional scFv-NKG2D fusion protein is capable of killing tumor cells in a specific manner without killing normal tissues/animals.

### EXAMPLE 5

### Production and Characterization of NKp30-scFv

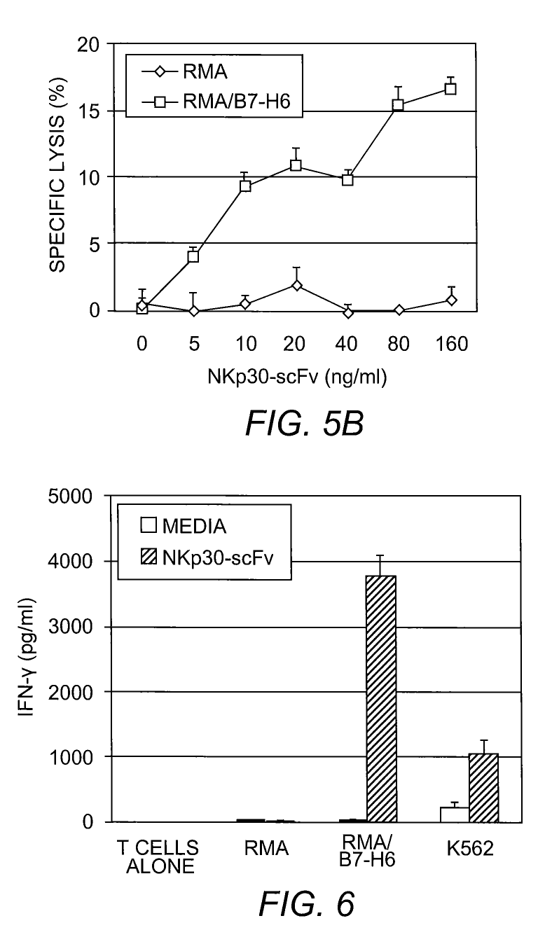

FIG. 5 shows data with another NK receptor scFv, NKp30-scFv. The data presented in FIG. 5A show cytotoxicity of RMA and RMA-B7/H6. B7-H6 is the ligand for NKp30, and only the ligand-positive tumor cells were killed. FIG. 5B is a cytotoxicity dose response with the NKp30-scFv.

FIG. 6 shows IFN-y production after co-culture of activated T cells and tumor cells. Use of NKp30-scFv resulted in specific IFN-γ production when ligand-positive tumor cells were present.

What is claimed is:

1. A monomeric bi-specific fusion protein comprising an effector cell-specific antibody fragment operably linked to at least a portion of a natural killer cell receptor, wherein said antibody fragment consists of the variable region of said antibody.

2. The fusion protein of claim 1, wherein the portion of the natural killer cell receptor comprises at least a portion of the extracellular domain.

3. The fusion protein of claim 1, wherein the NK cell receptor is selected from the group of NKG2D, NKG2A/CD94, NKRP1, NKG2C/CD94, NKG2E/CD94, NKG2F/CD94, NKp30, NKp44, NKp46, DNAM-1, CD69, LLT1, AICL, and CD26.

4. The fusion protein of claim 1, wherein the effector cell-specific antibody fragment binds an activating receptor expressed on a T cell, NK cell, macrophage, dendritic cell, or neutrophil.

5. The fusion protein of claim 4, wherein the activating receptor is selected from the group of CD3, CD4, CD8, CD16, CD28, CD16, NKp30, NKp44, NKp46, mannose receptor, CD64, scavenger receptor A, and DEC205.

6. The fusion protein of claim 1, wherein the effector cell-specific antibody fragment is operably linked to the at least a portion of a natural killer cell receptor via a linker.

7. A pharmaceutical composition comprising the fusion protein of claim 1 in admixture with a pharmaceutically acceptable carrier.

8. The pharmaceutical composition of claim 7, further comprising at least one second therapeutic agent.

9. A nucleic acid molecule encoding the fusion protein of claim 1.

10. A vector comprising the nucleic acid molecule of claim 9.

11. A bacterial host cell comprising the vector of claim 10.

12. A mammalian host cell comprising the vector of claim 10.

13. A method for treating cancer comprising administering to a subject in need of treatment an effective amount of the fusion protein of claim 1 so that the subject's cancer is treated.

14. A method for preventing cancer development or progression comprising administering to a subject with precancerous lesions or predisposition to cancer an effective amount of the fusion protein of claim 1 so that the subject's cancer is prevented.

15. A method for enhancing immunity against a tumor comprising administering to a subject in need of treatment an effective amount of the fusion protein of claim 1 so that immunity to the subject's tumor is enhanced.

16. The method of claim 15, further comprising administering one or more anti-cancer agents.

17. A method for treating a pathogen infection comprising administering to a subject in need of treatment an effective amount of a fusion protein of claim 1 so that the subject's pathogen infection is treated.


SEQUENCE LISTING (10 sequences)

SEQ ID NO: 1 - protein, 5 aa, Artificial sequence
  Synthetic peptide
SFPMA

SEQ ID NO: 2 - protein, 17 aa, Artificial sequence
  Synthetic peptide
TISTSGGRTYYRDSVKG

SEQ ID NO: 3 - protein, 10 aa, Artificial sequence
  Synthetic peptide
FRQYSGGFDY

SEQ ID NO: 4 - protein, 13 aa, Artificial sequence
  Synthetic peptide
TLSSGNIENNYVH

SEQ ID NO: 5 - protein, 7 aa, Artificial sequence
  Synthetic peptide
DDDKRPD

SEQ ID NO: 6 - protein, 9 aa, Artificial sequence
  Synthetic peptide
HSYVSSFNV

SEQ ID NO: 7 - protein, 134 aa, Artificial sequence
  Synthetic peptide
FNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 8 - protein, 171 aa, Artificial sequence
  Synthetic peptide
QSKAQVLQSVAGQTLTVRCQYPPTGSLYEKKGWCKEASALVCIRLVTSSKPRTMAWTSRFTIWDDPDAGFFTVTMTDLREEDSGHYWCRIYRPSDNSVSKSVRFYLVVSPASASTQTSWTPRDLVSSQTQTQSCVPPTAGARQAPESPSTIPVPSQPQNSTLRPGPAAPIA

SEQ ID NO: 9 - protein, 148 aa, Artificial sequence
  Synthetic peptide
KNSFTKLSIEPAFTPGPNIELQKDSDCCSCQEKWVGYRCNCYFISSEQKTWNESRHLCASQKSSLLQLQNTDELDFMSSSQQFYWIGLSYSEEHTAWLWENGSALSQYLFPSFETFNTKNCIAYNPNGNALDESCEDKNRYICKQQLI

SEQ ID NO: 10 - protein, 15 aa, Artificial sequence
  Synthetic peptide
GGGGSGGGGSGGGGS